(19) United States
(12) Patent Application Publication
(10) Pub. No.: US 2007/0190122 A1
(43) Pub. Date: Aug. 16, 2007

(54) Skin dressings

(76) Inventors: Paul James Davis, Felmersham (GB); Andrew John Austin, Irchester (GB); Jan Jezek, Stanwick (GB)

Correspondence Address: MORGAN LEWIS & BOCKIUS LLP, 1111 PENNSYLVANIA AVENUE NW, WASHINGTON, DC, 20004, US

(21) Appl. No.: 10/587,547

(22) PCT Filed: Jan. 28, 2005
(86) PCT No.: PCT/GB05/00284

§ 371 Date: Jul. 28, 2006

(30) Foreign Application Priority Data
Jan. 30, 2004 (EP) 04250508.1

(51) Int. Cl. A61L 15/00; A61K 38/44

(52) U.S. Cl. 424/445; 424/94.4

(57) ABSTRACT

A skin dressing of the general form disclosed in WO 03/030800 comprises oxidoreductase enzyme in hydrated condition in a hydrated hydrogel of hydrophilic polymer material, wherein hydrogel comprises at least 25% by weight of the polymer material. A currently preferred dressing comprises a lower, skin-contacting layer (18) comprising a hydrated hydrogel comprising 30% by weight sodium poly-AMPS and 5% by weight glucose, and an upper layer (16) comprising a hydrated hydrogel comprising 15% by weight sodium poly-AMPS, 15% by weight ammonium poly-AMPS, and glucose oxidase. Using hydrogels with a higher concentration of polymer material is found to affect the rate of oxygen generation and hence the oxygen concentration profile beneath the dressing in use in a manner beneficial to wound healing.

### FIELD OF THE INVENTION

This invention relates to skin dressings for application to a part of a human or animal body for treatment of skin, and relates particularly (but not exclusively) to wound dressings for treatment of compromised skin, particularly skin lesions, i.e. any interruption in the surface of the skin, whether caused by injury or disease, including skin ulcers, burns, cuts, punctures, lacerations, blunt traumas, acne lesions, boils etc.

### BACKGROUND TO THE INVENTION

WO 03/090800 discloses a skin dressing comprising oxidoreductase enzyme, e.g. glucose oxidase, in a hydrated hydrogel, e.g. of hydrophilic polymer material, with one preferred polymer being poly 2-acrylamido-2-methylpropane sulphonic acid (poly-AMPS) or salts thereof (e.g. as described in WO 01/96422). The dressing may include a source of substrate for the oxidoreductase enzyme, β-D glucose in the case of glucose oxidase. For example, FIG. 6 of WO 03/090800 discloses a skin dressing comprising a lower, skin-contacting layer including 20% by weight sodium poly-AMPS and 20% by weight glucose (substrate), and an upper layer in the form of a film of polyvinyl alcohol (PVA) incorporating glucose oxidase (enzyme).

The dressings of WO 03/090800 are used by being located on the skin of a human or animal, e.g. over a wound or on a region of skin to be treated for cosmetic or therapeutic purposes, e.g. for treatment of acne or other skin conditions. The oxidoreductase enzyme catalyses a reaction of an appropriate substrate with oxygen to produce hydrogen peroxide in a controlled manner in the dressing. The hydrogen peroxide diffuses through the dressing to the dressing/skin interface, where it has beneficial effects, e.g. being converted to oxygen by the enzyme catalase which is naturally present in wounds. Oxygen produced in this location inhibits anaerobic bacteria and supports the essential metabolism of cells engaged in the healing process.

We have now found that the profile of oxygen concentration within the space under the dressing (equivalent to the wound bed) follows a predictable profile, starting with a period of oxygen depletion, caused by the freshly placed dressing blocking the supply of atmospheric oxygen. This is followed by a sustained rise in oxygen, as the dressing starts to transmit oxygen via the diffusion of hydrogen peroxide, generated in-situ. Subsequently, the oxygen level reaches a plateau at saturation, and in the longer term (depending on the stage of the wound), gradually declines.

We have also found, surprisingly, that the time course of this profile varies greatly in proportion to the concentration of polymer within the gel, even though all other (active) ingredients remain the same (other than water, of course). The discovery of this effect now allows us to design dressings in which we can control the oxygen delivery profile to match the needs of a wound bed.

In particular, we have found that it is beneficial to use a hydrated hydrogel with a higher concentration of polymer material and a lower concentration of water than those specifically disclosed in WO 03/090800 for the enzyme-containing gel at least, and preferably also for a separate substrate-containing gel.

WO 97/02811 discloses a polymeric hydrogel patch including glucose oxidase, for application to the skin to measure glucose levels. Glucose drawn from the skin is converted in the patch to hydrogen peroxide that reacts at an electrode surface remote from the skin to generate an electrical signal related to the amount of glucose entering the patch. The patch functions as a diagnostic measurement patch, and is not a skin dressing, having no treatment effect on skin.

### SUMMARY OF THE INVENTION

The present invention provides a skin dressing comprising a first hydrated hydrogel of hydrophilic polymer material containing oxidoreductase enzyme in hydrated condition, wherein the hydrogel comprises at least 25% by weight of the polymer material.

The dressing preferably comprises a separate, second hydrated hydrogel of hydrophilic polymer material containing a source of substrate for the oxidoreductase enzyme, the hydrogel comprising at least 25% by weight of the polymer material.

The or each hydrogel is preferably in the form of a solid layer, sheet, slab or film of material that is typically cross-linked, and that may incorporate a mechanical reinforcing structure. The size and shape of the layer, sheet, slab or film can be selected to suit the intended use of the dressing. Thicknesses in the range 0.01 to 1.0 mm, preferably 0.05 to 0.5 mm are particularly suitable.

Alternatively, the or each hydrated hydrogel may be in the form of an amorphous gel not having a fixed form or shape, that can be deformed and shaped in three dimensions, including being squeezed through a nozzle. Amorphous gels are typically not cross-linked or have low levels of cross-linking. A shear-thinning amorphous gel may be used. Such a gel is liquid when subjected to shear stress (e.g. when being poured or squeezed through a nozzle) but set when static. Thus the gel may be in the form of a pourable or squeezable component that may be dispensed, e.g. from a compressible tube or a syringe-like dispenser, comprising a piston and cylinder, typically with a nozzle of about 3 mm diameter. Such a gel may be applied in the form of a surface layer, or into a wound cavity as a fully conformable gel that fills the available space and contacts the wound surface.

The dressing is thus preferably of layered construction, with the first hydrogel (including enzyme) constituting an upper layer (to be located remote from the skin in use) and the second hydrogel (including substrate) constituting a lower layer (to be located in contact with the skin in use).

Thus, in a preferred aspect the invention provides a skin dressing comprising: an upper layer (to be located remote from the skin in use) comprising a first hydrated hydrogel of hydrophilic polymer material containing oxidoreductase enzyme in hydrated condition, wherein the first hydrogel comprises at least 25% by weight of the polymer material; and a lower layer (to be located in contact with the skin in use) comprising a second hydrated hydrogel of hydrophilic polymer material containing a source of substrate for the oxidoreductase enzyme, wherein the second hydrogel comprises at least 25% by weight of the polymer material.

The or each hydrogel preferably comprises at least 30% by weight of the polymer material, and may comprise higher amounts, e.g. at least 40% by weight of the polymer material.

A hydrated hydrogel means one or more water-based or aqueous gels, in hydrated form.

A hydrated hydrogel can act to absorb water and other materials exuded from a wound site, enabling the dressing to perform a valuable and useful function by removing such materials from a wound site. The hydrated hydrogel also provides a source of moisture, that can act in use to maintain a wound site moist, aiding healing.

The hydrated hydrogel conveniently comprises hydrophilic polymer material. Suitable hydrophilic polymer materials include polyacrylates and methacrylates, e.g. as supplied by First Water Ltd in the form of proprietary hydrogels, including poly 2-acrylamido-2-methylpropane sulphonic acid (polyAMPS) or salts thereof (e.g. as described in WO 01/96422), polysaccharides e.g. polysaccharide gums particularly xanthan gum (e.g. available under the Trade Mark Keltrol), various sugars, polycarboxylic acids (e.g. available under the Trade Mark Gantrez AN-169 BF from ISP Europe), poly(methyl vinyl ether co-maleic anhydride) (e.g. available under the Trade Mark Gantrez AN 139, having a molecular weight in the range 20,000 to 40,000), polyvinyl pyrrolidone (e.g. in the form of commercially available grades known as PVP K-30 and PVP K-90), polyethylene oxide (e.g. available under the Trade Mark Polyox WSR-301), polyvinyl alcohol (e.g. available under the Trade Mark Elvanol), cross-linked polyacrylic polymer (e.g. available under the Trade Mark Carbopol EZ-1), celluloses and modified celluloses including hydroxypropyl cellulose (e.g. available under the Trade Mark Klucel EEF), sodium carboxymethyl cellulose (e.g. available under the Trade Mark Cellulose Gum 7LF) and hydroxyethyl cellulose (e.g. available under the Trade Mark Natrosol 250 LR).

Mixtures of hydrophilic polymer materials may be used in a gel.

The polymer material preferably comprises poly-AMPS or salts thereof.

Particularly good results have been obtained with an upper layer enzyme-containing first hydrogel comprising 15% by weight sodium poly-AMPS and 15% by weight ammonium poly-AMPS, and a lower layer substrate-containing second hydrogel comprising 30% by weight sodium poly-AMPS.

The dressing may otherwise be generally as disclosed in WO 03/090800.

The second hydrogel optionally incorporates a source of iodide ions (e.g. in the form of potassium iodide or sodium iodide) for reaction with hydrogen peroxide to generate molecular iodine, as disclosed in WO 03/090800. The second hydrogel preferably incorporates a source of zinc ions and/or a source of lactate ions (e.g. in the form of zinc lactate), as disclosed in WO 2004/108917 and preferably also glucose as these materials are thought to have beneficial effects on skin.

The currently preferred enzyme is glucose oxidase, with the corresponding substrate being glucose. Glucose is conveniently present in lower concentration than envisaged in WO 03/090800, e.g. constituting 5% by weight of the associated hydrogel: it has been found that greater amounts are superfluous and unnecessary.

A currently preferred dressing in accordance with the invention thus comprises an upper layer comprising a first hydrated hydrogel comprising 15% by weight sodium poly-AMPS, 15% by weight ammonium poly-AMPS, and glucose oxidase; and a lower, skin-contacting layer comprising a second hydrated hydrogel comprising 30% by weight sodium poly-AMPS and 5% by weight glucose.

The dressing is used by being located on the skin of a human or animal, e.g. over a wound or on a region of skin to be treated for cosmetic or therapeutic purposes, e.g. for treatment of acne or other skin conditions. The second gel (the skin-contacting gel containing substrate) is placed in contact with the skin, and the first gel (the upper gel, containing enzyme) is located on top of the first gel. The dressing functions in use to produce hydrogen peroxide in the first gel or at the interface between the gels, with the hydrogen peroxide diffusing through the second gel and reacting to generate oxygen (in dissolved form) at the skin or wound surface, catalysed by catalase present at the wound surface and in wound fluid, as is explained on pages 14 and 15 of WO 03/090800. The effective transport of oxygen across the dressing in this way is very important and has beneficial effects for healing.

The generation of oxygen achieved with dressings of the invention is based on two consecutive chemical reactions occurring in the two gels that constitute stratified layers, as follows:

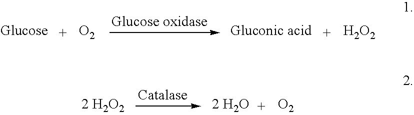

It is important that the reactions are spatially separated in the system, with the first one in the upper gel (away from the wound surface) and the second in the lower gel or at the wound contact surface of the lower gel.

In the first reaction, glucose from the lower, second gel diffuses into the upper, first gel and reacts with oxygen from the surrounding atmosphere, catalysed by the enzyme glucose oxidase trapped in the second gel, resulting in production of hydrogen peroxide. The hydrogen peroxide diffuses through the lower gel, and in the second reaction undergoes a reaction catalysed by catalase present at the skin surface and in wound fluid, resulting in production of oxygen. Oxygen produced in this way has beneficial effects, including inhibiting anaerobic bacteria and supporting the essential metabolism of cells engaged in the healing process.

Using hydrogels with a higher concentration of polymer material is found to affect the rate of internal hydrogen peroxide production and hence the oxygen concentration profile beneath the dressing in use in a manner beneficial to wound healing. In particular it results in an initial period of hypoxia (absence of oxygen) after location of the dressing on a surface that, surprisingly, is beneficial. We believe that, in general, there can be a need to have a period of hypoxia, during which time cells are stimulated to produce a cytokine called “hypoxia induced factor” (HIF). This triggers a cascade of cell signalling and biochemical responses that combine to bring about the process of neovascularisation, i.e. the formation of new blood vessels, central to the healing process. HIF production is considered to be crucial to the whole healing process, and our investigations have shown that a suitably prolonged period of hypoxia is beneficial.

The initial period of hypoxia is followed by a phase of oxygen generation at the interface between wound and dressing, resulting in an oxygen surge, until a saturated oxygen concentration is reached and maintained for a period of time. This is beneficial for wound healing. In particular, it is understood that wounds benefit by experiencing a period (or periods) of high oxygen concentration, to accelerate cell metabolism, provide white blood cells with high oxygen levels through which to enhance their antimicrobial biochemistry (respiratory burst) and to inhibit or eliminate pathogenic anaerobic bacteria.

Finally, it is also clear that a saturated oxygen concentration should not be maintained indefinitely, so any system of oxygenation should provide a longer term steady state (over days) of relatively low oxygen supply, or otherwise be readily controllable. This is inevitably achieved with a dressing in accordance with the invention, either by its tendency to suppress itself while in a dry state, or by its steady swelling (and dilution) on contact with an exuding wound, or through the simple process of being deliberately changed by the user at appropriate times. In this latter case, the patient or carer can control oxygen delivery to the wound by utilising the predictable delivery profile of the dressing to intervene at defmed time points, so as to tailor an oxygen delivery profile according to a treatment plan. This exploits the single-use, disposable nature of the dressing of this invention.

The present invention is based on the following observations and conclusions:
The concentration of the polymer, e.g. poly-AMPS, has a considerable effect on the rate of changes in oxygen concentration beneath the dressing, thus permitting dressings to be designed to deliver different oxygen profiles, according to the needs of different wounds.
The duration of the initial period of hypoxia following the application of the dressing increases with increasing concentration of polymer, e.g. poly-AMPS, in the dressing.
The rate of subsequent increase in oxygen concentration beneath the dressing is indirectly proportional to the polymer, e.g. poly-AMPS, concentration. The time required to achieve complete oxygenation (i.e. dissolved oxygen concentration equivalent to a solution equilibrated with pure gaseous oxygen) beneath the dressing is thus longer when using a high concentrated poly-AMPS dressing than when using lower concentration poly-AMPS dressings.

The overall conclusion is that there is in general considerable benefit in using a dressing incorporating one or more hydrogels comprising about 30% by weight poly-AMPS or salts thereof as the initial period of hypoxia in use of the dressings is extended to a highly advantageous degree.

Further beneficial effects of use of higher polymer, lower water content hydrogels are that the gels are more robust and easier to handle, and also retain structural integrity over time and so are less likely to leave debris at a wound site after use. The hydrogels also have higher water absorption properties.

Dressings in accordance with the invention (or components thereof, particularly individual hydrogels) are suitably supplied in sterile, sealed, water-impervious packages, e.g. laminated aluminium foil pouches.

Dressings in accordance with the invention can be manufactured in a range of different sizes and shapes for treatment of areas of skin, e.g. wounds, of different sizes and shapes. Appropriate amounts of enzyme, and substrate and iodide if present, for a particular dressing can be readily determined by experiment.

In a further aspect, the invention provides a method of producing a skin dressing comprising a first hydrated hydrogel of hydrophilic polymer material containing oxidoreductase enzyme in hydrated condition, comprising selecting the amount of polymer material so that the dressing in use produces oxygen at the skin surface at a desired rate.

The invention also includes within its scope a method of treating skin, comprising applying to the skin a skin dressing comprising a first hydrated hydrogel of hydrophilic polymer material containing oxidoreductase enzyme in hydrated condition, wherein the amount of polymer material in the first hydrated hydrogel is selected so that the dressing produces oxygen at the skin surface at a desired rate. The method may be used for cosmetic treatment of skin, as well as medical treatment of skin.

Preferred features of the dressing are as specified above, with the dressing preferably comprising upper and lower layers, with the amount of polymer material in the upper layer and in the lower layer being selected to produce oxygen at the skin surface at the desired rate.

The invention will be further described, by way of illustration, in the following Examples and with reference to the accompanying drawings in which:

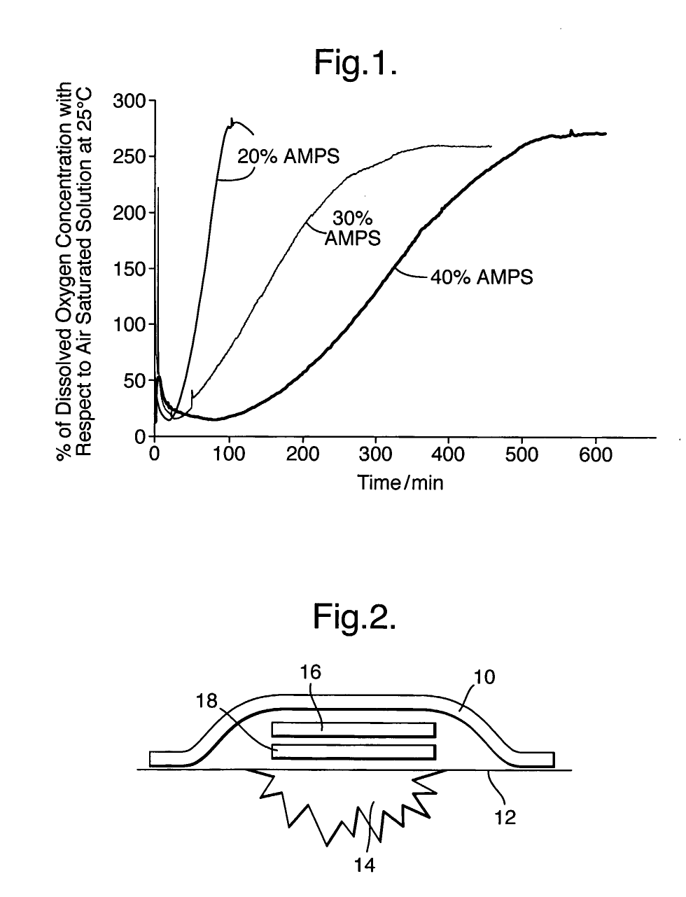

FIG. 1 is a graph of % of dissolved oxygen concentration with respect to air saturated solution at 25° C. versus time (in minutes), showing the rate of oxygenation of a hydrogel/sensor interface as a function of poly-AMPS concentration; and

FIG. 2 is a schematic sectional illustration of an embodiment of wound dressing in accordance with the invention.

### EXAMPLES

### Example 1

Experiments were carried out using the following materials:
Sodium AMPS—Lubrizol, code 2405
Glucose—Fisher—analytical grade, code G050061
Potassium iodide—Fisher—analytical grade, code P584050
1-hydroxy cyclo hexyl phenyl ketone (99%)—Aldrich—40,561-2 (this substance is referred to as ‘photoinitiator’)
Ebecryl 11 (PEG 400 diacrylate)—UCB Chemicals (this substance is referred to as ‘cross-linker’)
Glucose Oxidase—Biocatalysts—G638P (about 70 kU/gram powder)
Zinc L-lactate, hydrate—Aldrich
   Gel Preparation

The components were mixed in the combinations and quantities set out in Table 1, following the basic procedure set out below.

Stock solutions (as supplied by the manufacturer) of sodium AMPS were dispensed into a 250 ml polypropylene, screw-top reaction jar as the basis of the pre-gel fluid. Glucose oxidase (in the case of the top gel) and glucose, potassium iodide and zinc L-lactate (in the case of the lower or base gel) were added to the mixture and allowed to dissolve completely. In a separate vessel the photoinitiator powder was dispersed in the liquid cross-linker and the mixture was warmed gently to dissolve the photoinitiator into the cross-linker. This solution was then mixed into the pre-gel fluid. To cast the gels, the complete pre-gel fluid was poured into a flat bottomed tray, to a depth of 1-2 mm. The gels were set by UV irradiation from a 1 kW lamp, at a vertical distance of 15 cm, for 25 seconds. The gels were allowed to cool before use.

TABLE 1
Composition of hydrogels used in the study.

| | | |
|---|---|---|
| | Concentration of the | Concentration in the |
| Component | stock solution (w/w) | final gel (w/w) |

Components used in the top (enzyme) gels (first gels)

| | | |
|---|---|---|
| Na AMPS | 50% aq | 20% or 30% or 40% |
| Cross-linker | undiluted | 0.20% |
| Photoinitiator | undiluted | 0.01% |
| Glucose oxidase | solid powder | 90 μg/g |
| Water | | to total weight |

Components used in the base gel (second gels)

| | | |
|---|---|---|
| Na AMPS | 50% aq | 20% or 30% or 40% |
| Glucose | solid powder | 20% |
| Potassium iodide | 10% aq | 0.05% |
| Zinc L-lactate | 5% aq | 0.1% |

Active Oxygenation Monitoring

A chronoamperometric technique using specially modified screen-printed sensors was adopted to monitor the concentration of dissolved oxygen. Sensors were printed on an alumina substrate. Carbon paste (ED5000 from Electra Ltd, UK) was used to print the working electrode, the counter electrode and the connector tracks; Ag—AgCl paste was used to print the reference electrode. The working area of the sensors was covered tightly with a 0.005″ (0.013 mm) Teflon (Teflon is a Trade Mark) layer (Fluorocarbon) with the inner electrolyte (sodium phosphate, pH 6, 0.1 M; containing KCl, 0.1M) entrapped between the sensor surface and the Teflon layer.

The principle of the technique was identical to that of the commercially available ‘Clark oxygen sensors’. Dissolved oxygen diffuses through the Teflon layer into the electrode electrolyte where it is reduced at a working electrode poised at −550 mV vs. the Ag—AgCl reference electrode. The resulting cathodic current is proportional to concentration of dissolved oxygen.

Active oxygenation was monitored at the hydrogel/sensor interface. This was to mimic the processes occurring in vivo at the wound/dressing interface. A piece (approximately 2.5×2.5 cm) of the base gel layer was placed onto the surface of the sensor. 20 μL of electrode buffer containing catalase (100 μmL⁻¹) was placed between the sensor and the base layer. The system was activated by placing a piece (approximately 1.5×1.5 cm) of the enzyme gel layer onto the base layer and dissolved oxygen concentrations were monitored beneath the base layer (i.e. at the hydrogel/sensor interface).

Results and Discussion

Three stages of oxygen concentration profile were observed at the sensor/hydrogel interface following application of the freshly activated hydrogel dressing onto the sensor. The system was activated at time 0 by bringing the two hydrogel layers together. Saturation of the electrochemical response in the region 250-300% on the y-axis corresponds to reaching the maximum oxygen solubility. This is in accord both with calibration data and with visual evidence: gas started evolving at the gel/sensor interface when the oxygenation values stabilised at the 250-300% mark on the y-axis. The poly-AMPS concentrations stated refer to both the base layer and the top layer. Glucose concentration in the base layer was 20% w/w and glucose oxidase concentration in the enzyme layer was 90 μg per gram of gel.

Results are shown in FIG. 1.

As shown in FIG. 1, the three stages of oxygen concentration profile are as follows:

1) First, there was a gradual decline in dissolved oxygen concentration reflecting the low solubility of oxygen in the hydrogel. The sensor interface was ‘suffocated’ by the hydrogel.

2) After some time (20-90 minutes depending on concentration of poly-AMPS) the concentration of oxygen started increasing. This was due to the delivery of hydrogen proxide to the dressing/sensor interface and its immediate breakdown to oxygen by the enzyme catalase.

3) Finally, when the concentration of the dissolved oxygen at the dressing/sensor interface reached saturation the electrochemical signal stabilised. Slow evolution of gas was observed at the dressing/sensor interface shortly after.

The time profile of the above processes observed at the dressing/sensor interface was found to be dependent upon the concentration of poly-AMPS in the hydrogels.

The duration of the initial decline in oxygen concentration (‘sensor suffocation’) increased with increasing concentration of poly-AMPS. This was due to the slower generation of hydrogen peroxide in the top hydrogel layer and subsequent slower diffusion of peroxide to the sensor interface. The time required for the oxygen delivery to start at the interface was thus longer with a more concentrated poly-AMPS hydrogel than with that using a less concentrated poly-AMPS hydrogel.

The subsequent rate of the increase of oxygen concentration was higher in the case of low poly-AMPS concentration than with higher concentrations. This was also caused by more rapid generation of peroxide in the top gel and its more rapid diffusion towards the sensor interface. Complete oxygenation (i.e. dissolved oxygen concentration equivalent to a solution equilibrated with pure gaseous oxygen) could be achieved at the dressing/sensor interface in approximately 50 minutes following application of the top gel layer using 20% poly-AMPS, in approximately 300 minutes using 30% poly-AMPS and in approximately 500 minutes using 40% poly-AMPS (FIG. 1).

### Example 2

FIG. 2 illustrates schematically a skin dressing in accordance with the invention.

The illustrated dressing is of layered construction and comprises an outer layer or covering 10 in the form of an oxygen-permeable self-adhesive plaster, suitable for adhering to the skin 12 of a subject, so as to cover a wound 14. Covering 10 encloses an upper layer 16 comprising a second hydrogel and a lower layer 18 comprising a first hydrogel.

The second hydrogel comprises a layer of a poly-AMPS hydrogel that incorporates glucose oxidase enzyme, as described below. The first hydrogel comprises a layer of poly-AMPS hydrogel incorporating glucose, as described below.

The second hydrogel of lower layer 18 was formulated to include the following ingredients by weight:

| | |
|---|---|
| Water (ex Fisher, distilled, de-ionised, analytical grade) | 64.7% |
| Sodium AMPS (ex Lubrizol AMPS 2405 Monomer) | 30.0% |
| Polyethylene glycol diacrylate (PEG400 diacrylate, | 0.19% |
| ex UCB Chemicals available as Ebecryl 11) | |
| 1-hydroxycyclohexyl phenyl ketone (a photoinitiator, | 0.01% |
| ex Aldrich) | |
| Anhydrous glucose (enzyme substrate, ex Fisher) | 5.00% |
| Potassium iodide (ex Fisher) | 0.05% |
| Zinc L-lactate hydrate (ex Aldrich) | 0.10% |

The mixture was dispensed into casting trays containing either polyester scrim (polyester non-woven, open mesh support, available from HDK Industries Inc, Product Code 5722) or polyethylene net support, of dimensions 100 mm×100 mm, to a depth of about 1.5 mm. The polyethylene net support was fabricated from polyester staple fibres thermally bonded by a polyester resin—Product code 5722, from Castle Industries, Greenville, S.C. 9609, USA. The hydrogel was then set, by irradiation under a UV lamp, for up to 60 seconds and a power rating of approximately 100 mW/cm². The hydrogel was then allowed to cool to 30° C. or below.

The enzyme-containing first hydrogel of upper layer 16 was formulated to include the following ingredients by weight:

| | |
|---|---|
| Water (ex Fisher, distilled, de-ionised, analytical grade) | 68.6% |
| Sodium AMPS (ex Lubrizol AMPS 2405 Monomer) | 15.0% |
| Ammonium AMPS (ex Lubrizol AMPS 2411 Monomer) | 15.0% |
| Polyethylene glycol diacrylate (PEG400 diacrylate, | 0.19% |
| ex UCB Chemicals available as Ebecryl 11) | |
| 1-hydroxycyclohexyl phenyl ketone (a photoinitiator, | 0.01% |
| ex Aldrich) | |
| Glucose oxidase (GOX, Biocatalysts, Pontypridd, | 0.035% |
| Code G575P) | |
| Zinc L-lactate hydrate (ex Aldrich) | 1.0% |
| Pluronic P65 (block co-polymer of ethylene oxide | 0.15% |
| and propylene oxide, HO—[CH2CH2O]x- | |
| [CH2CHCH3O]y-[CH2CH2O]y-H, average | |
| MW 3400 (BASF) | |

The mixture was dispensed into casting trays containing polyester scrim (polyester non-woven, open mesh support, available from HDK Industries Inc, Product Code 5722) of dimensions 100mm×100 mm, to a depth of about 1.0 mm. The hydrogel was then set, by irradiation under a UV lamp, for up to 30 seconds (typically 25 seconds), and a power rating of approximately 100 mW/cm². The hydrogel was then allowed to cool to 30° C. or below.

The enzyme-containing hydrogel and the glucose-containing hydrogel were bought together, one overlying the other.

An oxygen-permeable and moisture-permeable covering or overlay such as of polyurethane may be located over the enzyme-containing hydrogel and may be adhered to the skin by means of e.g. acrylic adhesive provided on the lower face of the overlay.

The resulting product was packaged in an oxygen-impermeable and water-impervious pouch or enclosure, e.g. made of laminated aluminium foil pouches as supplied by Sigma (code Z183407).

1. A skin dressing comprising a first hydrated hydrogel of hydrophilic polymer material containing oxidoreductase enzyme in hydrated condition, wherein the hydrogel comprises at least 25% by weight of the polymer material.

2. A dressing according to claim 1, further comprising a second hydrated hydrogel of hydrophilic polymer material containing a source of substrate for the oxidoreductase enzyme, the hydrogel comprising at least 25% by weight of the polymer material.

3. A dressing according to claim 2, wherein the first and second hydrogels are each in the form of a respective layer, sheet or slab.

4. A dressing according to claim 3, wherein the first hydrogel constitutes an upper layer of the dressing and the second hydrogel constitutes a lower layer of the dressing.

5. A dressing according to claim 4, wherein the first hydrogel comprises 15% by weight sodium poly-AMPS and 15% by weight ammonium poly-AMPS, and the second hydrogel comprises 30% by weight sodium poly-AMPS.

6. A dressing according to claim 5, wherein the substrate is glucose.

7. A dressing according to claim 6, wherein glucose constitutes 5% by weight of the second hydrogel.

8. A dressing according to claim 7, wherein the second hydrogel includes a source of iodide ions.

9. A dressing according to claim 8, wherein the second hydrogel includes a source of zinc ions and/or a source of lactate ions.

10. A dressing according to claim 9, comprising a lower, skin contacting layer comprising the second hydrated hydrogel comprising 30% by weight sodium poly-AMPS and 5% by weight glucose; and an upper layer comprising the first hydrated hydrogel comprising 15% by weight sodium poly-AMPS, 15% by weight ammonium poly-AMPS, and glucose oxidase.

11. A dressing according to claim 2, wherein the enzyme is glucose oxidase.

12. A dressing according to claim 2, wherein the or each hydrogel comprises at least 30% by weight of the polymer material.

13. A dressing according to claim 2, wherein the polymer material of the or each hydrogel comprises poly-AMPS or salts thereof.

14. A method of producing a skin dressing comprising a first hydrated hydrogel of hydrophilic polymer material containing oxidoreductase enzyme in hydrated condition, comprising selecting the amount of polymer material so that the dressing in use produces oxygen at the skin surface at a desired rate.

15. A method of treating skin, comprising applying to the skin a skin dressing comprising a first hydrated hydrogel of hydrophilic polymer material containing oxidoreductase enzyme in hydrated condition, wherein the amount of polymer material in the first hydrated hydrogel is selected so that the dressing produces oxygen at the skin surface at a desired rate.
